(19) United States
(12) Patent Application Publication
(10) Pub. No.: US 2023/0255279 A1
(43) Pub. Date: Aug. 17, 2023

(54) THIGHS PROTECTOR

(71) Applicant: Abdallah ALASRAJ, Riyadh (SA)

(72) Inventor: Abdallah ALASRAJ, Riyadh (SA)

(21) Appl. No.: 17/670,008

(22) Filed: Feb. 11, 2022

(51) Int. Cl. A41D 13/05; A61F 13/06; A41D 31/18

(52) U.S. Cl. CPC A41D 13/0543; A41D 31/185; A61F 13/06

(57) ABSTRACT

A thighs protector is described. The thighs protector comprises an elongated adhesive skin dressing having a first side including an adhesive pattern and a second side including hydrophobic, friction free material. The elongated adhesive skin dressing is configured to cover at least a section of an inner thigh of a human. The elongated adhesive skin dressing has an outer edge which follows an outer extent of a sartorius muscle of the inner thigh and tapers from a first width near a top of the inner thigh to a second width at an inside of a knee. The first width is larger than the second width.

### BACKGROUND

### Technical Field

The present disclosure is directed to a thigh covering, and more particularly directed to a thighs protector in the form of a removeable adhesive skin dressing which is suitable to effectively prevent the occurrence of chafing on inner thigh surfaces of a person.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Physical activity may cause friction when one portion of a person’s skin rubs against another portion of skin. The most common examples of such skin to skin friction are the result of the inner surfaces of the thighs of a person rubbing against each other during physical activity, such as walking, running, hiking, mountain climbing or playing sports. Chafing can occur due to such skin friction, which can lead to rashes, irritation or even skin conditions like miliaria, for example, at the inner thigh region. Moreover, chafing is possible even when there is no physical movement. Chafing may occur in crowded environments when a large number of persons are standing and moving close to each other. For instance, in Saudi Arabia, crowds performing the rituals of Hajj and Umrah at mosques have reported suffering skin flaking and redness on their inner thighs. Other examples of crowded environments in which chaffing of the inner thighs can occur are concerts, music festivals, etc.

There are some remedies available in the market which may provide temporary relief to the affected areas, such as moisturizing creams. However, even with the use of moisturizing creams and similar products, the problem of skin exfoliation between the thighs ca occur as a result of physical exercise, crowding and extreme heat.

Solutions to the problem of protecting the inner thighs from chafing have been suggested. For example, DE202011110364 describes a thighs protector to prevent chafing on the inner surfaces while at the same time ensuring breathability in the area of the thighs protector, even at high temperatures. US6227937B1 relates to a thigh anti-chafing garment. However, in both references, the protective devices are not adhesive skin dressings but instead are in form of an elastic sleeve which wraps around the thigh and thus cannot be easily removed and replaced during an event.

US20070111868A1 describes a tights-type leg support garment, having inner leg protective sections which provide support to the sartorius muscle. The supported area appears to run along areas between the thighs. However, this reference is a garment, not an adhesive skin dressing.

A thigh tape sticker patch sold by AliExpress, “1 Pair Invisible Thigh Tape Sticker Patch Anti-Scratch Ultra Thin Sweat Leg Tapes Unisex Disposable Spandex Pads Foot Tool”, www.aliexpress.com/item/1005003053824522, describes an oval thigh tape to reduce friction between the legs, which is detachable and breathable on the skin, with soft, fabric material (Spandex) on the outer side and an oval shape, and claims to be skin-friendly and non-irritating to use. Although the adhesive is not described, the patch is a “tape” such as one using an adhesive. However, this patch is not shaped to follow the sartorius muscle along the inner thigh.

Each of the aforementioned references suffers from one or more drawbacks hindering their adoption. Accordingly, it is one object of the present disclosure to provide a thighs protector and a method of using a thighs protector, which tracks the sartorius muscle and is configured to expand when the sartorius muscles stretches and retract when the sartorius muscle relaxes.

### SUMMARY

In an exemplary embodiment, a thighs protector is described. The thighs protector comprises an elongated adhesive skin dressing having a first side including an adhesive pattern and a second side including hydrophobic, friction free material. The elongated adhesive skin dressing is configured to cover at least a section of an inner thigh of a human. The elongated adhesive skin dressing has an outer edge which follows an outer extent of a sartorius muscle of the inner thigh and tapers from a first width near a top of the inner thigh to a second width at an inside of a knee. Herein, the first width is larger than the second width.

In another exemplary embodiment, a thighs protector is described. The thighs protector comprises an elongated adhesive skin dressing configured to cover at least a section of an inner thigh of a human. The elongated adhesive skin dressing has an outer edge which follows an outer extent of a sartorius muscle of the inner thigh and tapers from a rounded first width within a first threshold distance from a groin at a top of the inner thigh to a second rounded width at an inside of the thigh, at a second threshold distance above a knee. Herein, the first width is larger as the second width.

In another exemplary embodiment, a method of using a thighs protector is described. The method comprises removing a peelable paper from the thighs protector, to reveal an adhesive side of an elongated adhesive skin dressing. The method further comprises identifying a first width and a second width of the elongated adhesive skin dressing, wherein the first width is larger than the second width. The method further comprises positioning the adhesive side with the adhesive above an inner thigh over a sartorius muscle of the inner thigh. The method further comprises aligning the adhesive side of the first width at a first threshold distance from a groin. The method further comprises pressing the adhesive side of the first width onto the thigh. The method further comprises pressing and smoothing the adhesive side of the elongated adhesive s towards a knee, such that the adhesive side adheres to the thigh along the sartorius muscle. The method further comprises pressing the adhesive side of the second width to align with an inside of a knee.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an exemplary diagrammatic illustration of a thighs protector from a first side thereof, according to a first embodiment.

FIG. 2 is an exemplary diagrammatic illustration of the thighs protector from a second side thereof, according to the first embodiment.

FIG. 3 is an exemplary diagrammatic illustration of the thighs protector of FIGS. 1-2 depicting its implementation on a thigh of a person, according to the first embodiment.

FIG. 4 is an exemplary diagrammatic illustration of the thighs protector depicting its placement with respect to sartorius muscle in the thigh of the person, according to the first embodiment.

FIG. 5 is an exemplary diagrammatic illustration of a thighs protector having a pattern of expansion bands, according to a second embodiment.

FIG. 6 is an exemplary diagrammatic illustration of the thighs protector of FIG. 5 depicting its placement with respect to sartorius muscle in the thigh of the person, according to the second embodiment.

FIG. 7 is an exemplary diagrammatic illustration of a thighs protector having a bumper, according to a third embodiment.

FIG. 8 is an exemplary diagrammatic illustration of the thighs protector of FIG. 7 depicting its placement with respect to sartorius muscle in the thigh of the person, according to the third embodiment.

FIG. 9A is an exemplary diagrammatic illustration depicting the effect between the thighs of a person without a thighs protector.

FIG. 9B is an exemplary diagrammatic illustration depicting a reduced friction between the thighs of a person wearing the thighs protector, according to certain embodiments.

FIG. 10 is an exemplary flowchart of a method of using a thighs protector, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a thighs protector and method of using a thighs protector for people who may need to walk long distances, perform a heavy physical routine involving walking/running, and/or moving in crowded environment. The thighs protector is used to protect the thighs from chafing as they move against each other during walking, running or standing for long periods of time, especially in crowded circumstances. The thighs protector includes an elongated adhesive skin dressing configured to cover an inner thigh, wherein the elongated adhesive skin dressing has an outer edge which follows an outer extent of the sartorius muscle and tapers from a first width near a top of the inner thigh to a second width at an inside of a knee, wherein the first width is larger than the second width.

The thighs protector may generally be shaped conforming to inner thigh. Embodiments include: (i) an adhesive pattern in which a stronger adhesive is on the periphery of the skin dressing and an adhesive with a lower tack strength is on the remainder of the skin dressing; (ii) one or more expansion bands to allow flexion of the thigh muscles, the expansion bands comprising one or more of a pleated material, an adhesive pattern with a low tack strength region and a region without adhesive, a pre-stressed material, a folded material and a tucked material; (iii) a bumper expending along a midline of the elongated adhesive skin dressing, the bumper including at least one of a thicker material, a plurality of layers of material and a filled channel. Further embodiments include an adhesive receiving surface on the first side of the skin dressing and a hydrophobic, friction free material on a second side of the elongated adhesive skin dressing.

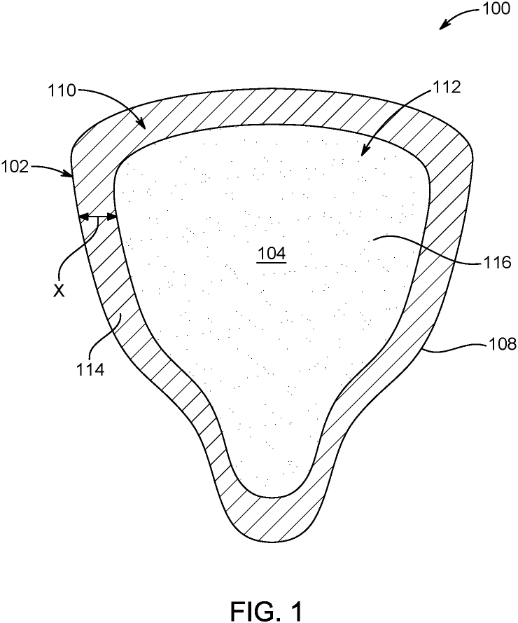

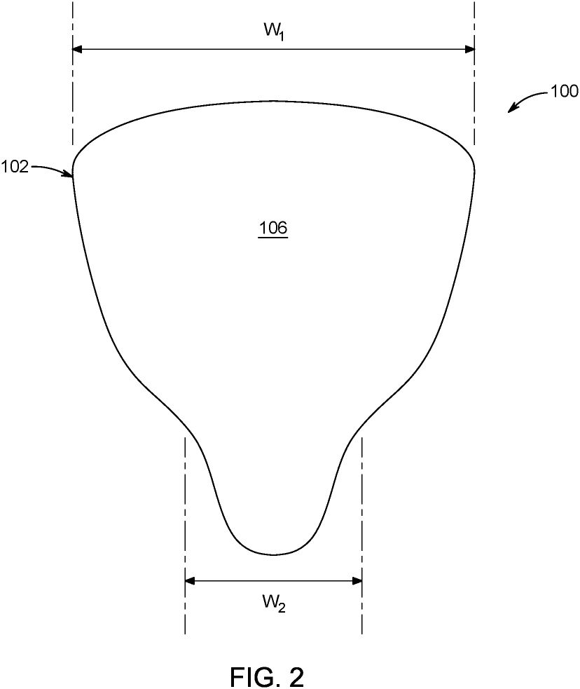

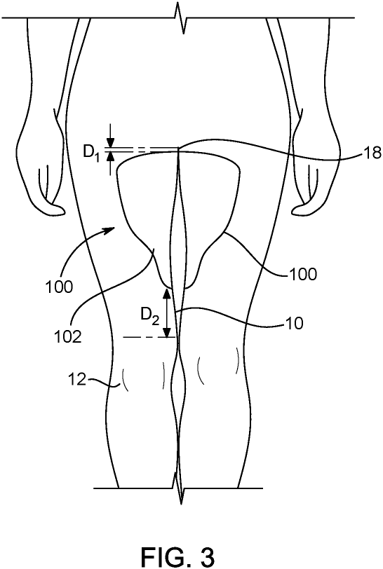

Referring to FIGS. 1 and 2 in combination, a thighs protector 100 is illustrated according to a first embodiment. The thighs protector 100 includes an elongated adhesive skin dressing 102, and may include a protective backing sheet (not shown) present on the surface of the adhesive side designed to be removed before use. In general, the elongated adhesive skin dressing 102 defines the shape of the thighs protector 100 which is designed to be applied to an inner thigh of a person. Hereinafter, the terms “elongated adhesive skin dressing 102” and the “thighs protector 100” have generally been interchangeably used without any limitations. The elongated adhesive skin dressing 102 has a first side 104 and a second side 106. In the accompanied illustrations, the elongated adhesive skin dressing 102 is shown from the first side 104 thereof in FIG. 1, and from the second side 106 thereof in FIG. 2. Specifically, the elongated adhesive skin dressing 102 is configured to cover at least a section of an inner thigh of a human. That is, the elongated adhesive skin dressing 102 may be shaped to conform to the inner thigh (as represented by reference numeral 10) of a human, as depicted in FIG. 3. Further, as depicted in FIG. 3, the elongated adhesive skin dressing 102, when applied to the inner thigh 10 (also referred to as “thigh 10” without any limitations), extends from near a top of the inner thigh 10 to at an inside of a knee (as represented by reference numeral 12).

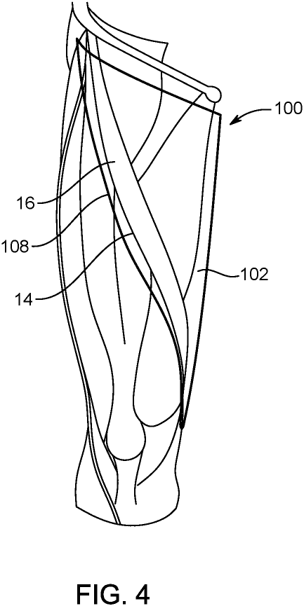

More specifically, as illustrated in FIGS. 1 and 2, the elongated adhesive skin dressing 102 has an outer edge 108. Herein, as better illustrated in FIG. 4, the outer edge 108 of the elongated adhesive skin dressing 102 follows an outer extent (as represented by reference numeral 14) of a sartorius muscle (as represented by reference numeral 16) of the inner thigh 10. As may be seen from FIG. 4, the sartorius muscle 16 has a generally arcuate profile. Thus, the outer edge 108 of the elongated adhesive skin dressing 102 also has a generally arcuate profile, conforming to the profile of the sartorius muscle 16. In particular, as may be understood from combination of FIGS. 1 - 3, the elongated adhesive skin dressing 102 tapers from a first width ‘W₁’ near the top of the inner thigh 10 to a second width ‘W₂’ at the inside of the knee 12. Herein, the first width ‘W₁’ is larger than the second width ‘W₂’. In one or more examples, the first width ‘W₁’ may be larger than the second width ‘W₂’ by about 30 - 60%. In a non-limiting example, the elongated adhesive skin dressing 102 may have the first width ‘W1’ in the range of 6 cm to 30 cm, preferably 10-20 cm. Further, the elongated adhesive skin dressing 102 may have the second width ‘W₂’ in the range of 4 cm to 20 cm, preferably 8-15 cm.

Further, in a non-limiting example, a vertical length (not labelled) of the elongated adhesive skin dressing 102 may be in range of 15 to 50 cm, preferably 25-40 cm and with a preferred size of 45 cm. This length is from the top edge of the adhesive skin dressing near the groin to the lower edge slightly above the knee. Such configuration imparts the arcuate shape to the outer edge 108 of the elongated adhesive skin dressing 102.

The first width ‘W₁’ should be approximately one fourth (e.g., 0.2-0.3 times) of the circumference of an upper thigh. The second width ‘W2’ should be approximately one fourth (e.g., 0.2-0.3 times) of the circumference of a lower thigh just above a knee. Consequently, the thighs protector 100 may be made available in a plurality of widths to accommodate users with different sizes of thigh circumferences. The first width ‘W₁’ should be at least four times, preferably 4 - 8 or about 6 times, greater than the second width ‘W₂’.

In an aspect of the present disclosure, as depicted in combination in FIGS. 3 and 4, the elongated adhesive skin dressing 102 tapers from the first width ‘W₁’ within a first threshold distance ‘D₁’ from a groin (as represented by reference numeral 18) at a top of the inner thigh 10 to the second width ‘W₂’ at a second threshold distance ‘D₂’ above the knee 12. That is, the elongated adhesive skin dressing 102 is placed below the groin 18 at the top of the inner thigh 10 and above the knee 12, while following the outer extent 14 of the sartorius muscle 16 of the inner thigh 10. In an example, the first threshold distance ‘D₁’ is selected to be one of 0 cm, 0.5 cm, and 1 cm or in the range of 1 cm to 8 cm. In an example, the second threshold distance ‘D₂’ is in a range of 2 cm to 8 cm, preferably 4-6 cm. Such placement of the elongated adhesive skin dressing 102 ensures that the thighs protector 100 is able to protect the inner thighs 10 from rubbing against each other, when the person may be walking or moving in a crowded environment.

In an aspect of the present disclosure, as may be seen from FIGS. 1-3, the elongated adhesive skin dressing 102 may have a rounded shape at the top of the inner thigh 10. That is, the elongated adhesive skin dressing 102 has a rounded shape at the top of the inner thigh 10 at the first threshold distance ‘D₁’ from the groin 18 at the top of the inner thigh 10. Further, in an aspect of the present disclosure, the elongated adhesive skin dressing 102 has a rounded shape at the second threshold distance ‘D₂’ above the knee 12. Such rounded shapes at top and bottom of the elongated adhesive skin dressing 102 allows for better conforming to the shape of the inner thigh 10 without causing irritation to upper region of the inner thigh 10 or to the knee 12. Alternatively, the elongated adhesive skin dressing 102 may have a straight shape or a convex shape at the top of the inner thigh 10 as shown in FIG. 4. The shape of the top of the elongated adhesive skin dressing is related to the distance ‘D₁’, and may be chosen for maximum conformance with the top of the thigh.

In one or more aspect of the present disclosure, the thighs protector 100 includes an adhesive receiving surface 110 on the first side 104 of the elongated adhesive skin dressing 102. That is, the elongated adhesive skin dressing 102 defines the adhesive receiving surface 110 in the first side 104 thereof. Specifically, as illustrated in FIG. 1, the first side 104 of the elongated adhesive skin dressing 102 defines an outer extent 110, which acts as the adhesive receiving surface. Herein, the outer extent 110 may extend up to a width (labelled as ‘X’ in FIG. 1) from the outer edge 108 of the first side 104 of the elongated adhesive skin dressing 102 to the inner extent 112. In an example, the width ‘X’ of the outer extent 110 from the outer edge 108 of the first side 104 may be in a range of 0.5 cm to 5 cm. Further, as illustrated, the first side 104 of the elongated adhesive skin dressing 102 defines an inner extent 112. Herein, the inner extent 112 is part of the inner region of the first side 104 of the elongated adhesive skin dressing 102. The outer extent 110 may include a first adhesive 114 and the inner extent 112 may include a second adhesive or may be free of adhesive.

In an aspect of the present disclosure, the thighs protector 100 includes a first adhesive 114 on the outer extent 110 of the first side 104 of the elongated adhesive skin dressing 102. With the outer extent 110 extending up to the width ‘X’ from the outer edge 108 of the first side 104, the first adhesive 114 may also extend up to the width ‘X’ from the outer edge 108 of the first side 104. Further, the thighs protector 100 may include a second adhesive 116 on the inner extent 112 of the first side 104 of the elongated adhesive skin dressing 102. In general, the second adhesive 116 may be uniformly applied on the entre inner region (as defined by the inner extent 112) of the first side 104 of the elongated adhesive skin dressing 102.

In aspects of the present disclosure, the second adhesive 116 has a lower tack strength than the first adhesive 114. The term “tack strength,” as used herein, is a measure of the ability of two materials to resist separation after bringing their surfaces into contact for a short time under a light pressure. In simpler terms, the tack strength may define an “adhesive power” of the adhesive being used. Methods of determining adhesive strength include ASTM D903 Peel or Stripping Strength of Adhesive Bonds 90 degree peel test data, ASTM F2256-05(2015) Standard Test Method for Strength Properties of Tissue Adhesives in T-Peel by Tension Loading and ASTM D3330 Peel Adhesion of Pressure Sensitive Tape; ASTM D1876 T-Peel Test. The peel strength of the skin dressing is preferably in a range of 7 Newtons/25 mm to 26 Newtons/25 mm. The tack strength of the dressing is preferably in the range of 3 Newtons to 10 Newtons.

Thus, the first adhesive 114 with higher tack strength may apply the elongated adhesive skin dressing 102 on the inner thigh 10 to conform to the shape of the sartorius muscle, while the second adhesive 116 with lower tack strength may provide some “stickiness” to hold the elongated adhesive skin dressing 102 on the inner thigh 10 without shifting. In an aspect, the first adhesive 114 and the second adhesive 116 are covered with a peelable paper (not shown). The peelable paper ensures that the first adhesive 114 and the second adhesive 116 may not be exposed or come in contact with any other element, such as packaging of the thighs protector 100, when the thighs protector 100 may not be in use, or specifically before first use of the thighs protector 100. It may be appreciated that the first adhesive 114 and the second adhesive 116 may be skin-safe, hypoallergenic, sweat-resistant, and non-toxic, since these may be in direct contact with the skin of the person using the thighs protector 100. In one or more aspects of the present disclosure, the first adhesive 114 is selected from the group comprising spirit gum, medical dermal glue, liquid latex, silicon rubber based adhesive, acrylate polymer resin, and the like. Further, the second adhesive 116 is selected from polyisoprene, polyisobutylene, and the like. ]In the present configuration, the low tack adhesive region, as formed by the second adhesive 116, is configured to release when the sartorius muscle 16 stretches and reattach when the sartorius muscle 16 relaxes, thus enabling the thighs protector 100 to be properly held against the inner thigh 10 even in case of expansion and contraction of the sartorius muscles 16, as is common during physical activity.

Also, in an aspect of the present disclosure, the thighs protector 100 includes a hydrophobic, friction free material on the second side 106 of the elongated adhesive skin dressing 102. Since the second side 106 of the elongated adhesive skin dressing 102 is the outer side of the thighs protector 100, such material may define a top layer for the thighs protector 100. The hydrophobic, friction free material for the second side 106 is selected from the group comprising silk-like material, polyester material, polypropylene (PP) fabric material, and the like. In one example, the second side 106 is made of silk-like material which is hydrophobic and has very low friction. This helps to minimize friction between the thighs protectors 100 on the opposing inner thighs of the person, when used. In some examples, the second side 106 may further be embedded with fragrance releasing particles. Also, it may be appreciated that the second side 106 may be made of antibacterial material, since it may be in direct contact with the skin of the person using the thighs protector 100. Further, the elongated adhesive skin dressing 102 may be provided with a pattern of air holes (not shown) from the first side 104 through the second side 106, specifically in the inner extent 112 thereof, to impart breathability characteristics to the thighs protector 100.

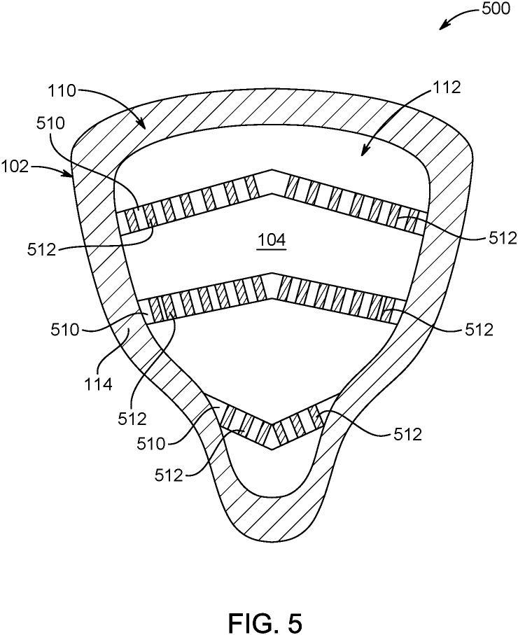

Referring now to FIGS. 5 - 6, in combination, illustrated is a thighs protector 500 according to a second embodiment. The thighs protector 500 is similar to the thighs protector 100 as described in the preceding paragraphs, with generally similar configurations for, but not limited to, the elongated adhesive skin dressing 102 with the first side 104, the outer edge 108, the outer extent 110 with the first adhesive 114, and the inner extent 112. Thus, the above-mentioned details for the said elements of the thighs protector 100 have not been repeated herein for the description of the thighs protector 500 for the sake of brevity of the present disclosure. Further, in the illustrations of FIGS. 5 - 6 for the thighs protector 500, some of the elements, as shown for the thighs protector 100 in FIGS. 1-4, have been omitted and some of the elements have not been labelled, for simplicity and brevity.

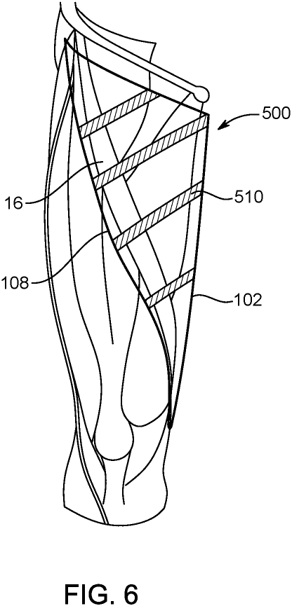

In the illustration of FIG. 5, the elongated adhesive skin dressing 102 of the thighs protector 500 is shown from the first side 104 thereof. As illustrated, the thighs protector 500 includes at least one expansion band 510 on the first side 104 of the elongated adhesive skin dressing 102. In the illustrations of FIG. 5, the thighs protector 500 is shown to include three expansion bands 510; however, it may be appreciated that the number of expansion bands 510 may be lower or higher without departing from the spirit and the scope of the present disclosure, e.g., 2-20 bands, 4-15 bands, or 6-10 bands. As illustrated, the expansion bands 510 may cross a width of the inner extent 112 of the elongated adhesive skin dressing 102. In the illustrated examples, the expansion bands 510 have been shown in the form of strips but it may be appreciated that the expansion bands 510 may have some other suitable shape such as a cylindrical cross-section or a barbell shape, without any limitations. FIG. 6 depicts placement of the thighs protector 500 with respect to sartorius muscle 16 in the inner thigh 10 of the person. As illustrated, the outer edge 108 of the elongated adhesive skin dressing 102 of the thighs protector 500 follows the sartorius muscle 16 of the inner thigh 10.

As illustrated in FIG. 5, each expansion band 510 includes sections 512 defined therein. Such sections 512 may be defined throughout a length of the expansion band 510, as extending across the width of the inner extent 112 of the elongated adhesive skin dressing 102. In an aspect of the present disclosure, each expansion band 510, for the respective sections 512, includes at least one of: a pleated material; an adhesive pattern with a low tack adhesive region and a region without adhesive; a pre-stressed material; a folded material; adhesive tracks; an elastic material; and a tucked material. In the present examples, the pleated material may allow the expansion band 510 to expand and/or contract easily as needed during physical exercise; the pre-stressed material may allow the expansion band 510 to contract when applied over the inner thigh 10 for proper fitting thereon; the folded material may allow the expansion band 510 to expand when applied over the inner thigh 10 for proper fitting thereon; the elastic material may allow expansion and contraction of the muscles beneath the thighs protector; the adhesive tracks may allow the expansion band 510 to be held on the inner thigh 10; and the tucked material may allow the expansion band 510 to expand and/or contract easily as required. It may be appreciated that different sections 512 in the expansion band 510 may be formed of different types of the materials as provided herein, without any limitations.

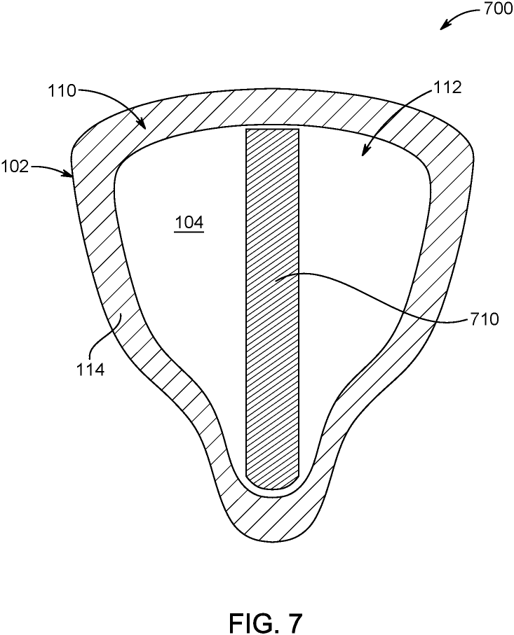

Referring now to FIGS. 7 - 8, in combination, a thighs protector 700 is illustrated according to a third embodiment. The thighs protector 700 is similar to the thighs protector 100 as described in the preceding paragraphs, with generally similar configurations for, but not limited to, the elongated adhesive skin dressing 102 with the first side 104, the outer edge 108, the outer extent 110 with the first adhesive 114, and the inner extent 112. Thus, the above-mentioned details for the said elements of the thighs protector 100 have not been repeated herein for the description of the thighs protector 700 for the sake of brevity of the present disclosure. Further, in the illustrations of FIGS. 7 - 8 for the thighs protector 700, some of the elements, as shown for the thighs protector 100 in FIGS. 1-4, have been omitted and some of the elements have not been labelled, for simplicity and brevity.

In the illustration of FIG. 7, the elongated adhesive skin dressing 102 of the thighs protector 700 is shown from the first side 104 thereof. As illustrated, the thighs protector 700 includes a bumper 710 extending to either side of a midline of the elongated adhesive skin dressing 102. As shown, the bumper 710 may extend vertically across a length of the inner extent 112 of the elongated adhesive skin dressing 102 and follow the sartorius muscle. Further, the bumper 710 may extend laterally at a middle of the inner extent 112 of the elongated adhesive skin dressing 102. In an example, a width of the bumper 710 may be in the range of 1 cm to 5 cm. In the present configuration, the bumper 710 may protrude perpendicularly from the first side 104 from the elongated adhesive skin dressing 102. In an aspect of the present disclosure, the bumper 710 includes at least one of: a thicker material; layers of material; and a filled channel. With such materials, the bumper 710 may act as a cushion when the thighs protector 700 is applied on the inner thigh 10 of the person. In an example, the filled channel may include hydroxyethyl cellulose, sodium polyacrylate, vinyl-coated silica gel, silicone and the like. Such gels may be chosen as these are generally non-toxic and thus may not cause any major harm in case the bumper 710 bursts.

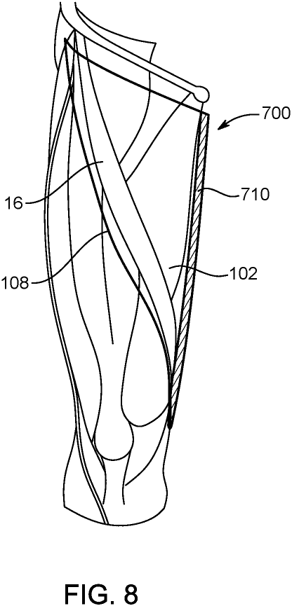

FIG. 8 depicts placement of the thighs protector 700 with respect to the sartorius muscle 16 on the inner thigh 10 of the person. As illustrated, the outer edge 108 of the elongated adhesive skin dressing 102 of the thighs protector 700 follows the sartorius muscle 16 of the inner thigh 10.

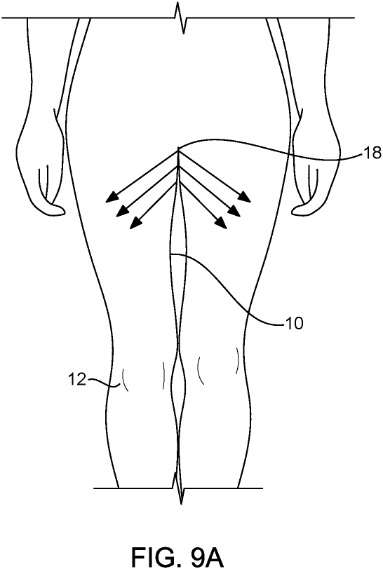

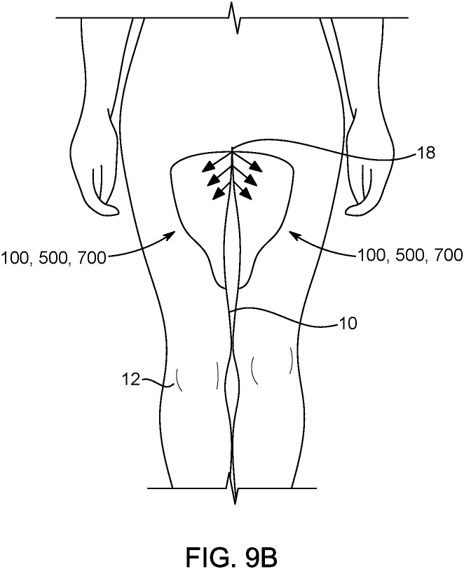

FIG. 9A depicts the effect of friction on the thighs of a person without the thighs protector and FIG. 9B depicts a decreased effect of friction on the thighs of a person with the thighs protector 100, 500, 700. As may be understood, most physical activity, such as walking, running, hiking, climbing, playing sports or even moving in a crowded environment, may cause rubbing of the inner thigh regions 10 between the knees 12 and the groin 18 of the person against each other. As depicted in FIG. 9A, such rubbing of the inner thigh regions 10 may result in skin friction (as depicted by arrows). This skin friction may, in turn, lead to rashes, irritation or even skin conditions like miliaria, for example, at the inner thigh region 10. As depicted in FIG. 9B, the use of the thighs protectors 100, 500, 700 applied at the inner thigh regions 10 may result in no or substantially reduced direct skin to skin rubbing at the inner thigh regions 10, and thus results in significantly reduced skin friction. In an example, the use of the thighs protectors 100, 500, 700 may result in skin friction being reduced by about 100% while standing still, 80%-90% while walking, and 60%-80% while running. Further, the use of the thighs protectors 100, 500, 700 may result in reduced irritation due to sweating at the inner thigh regions 10 by about 90%.

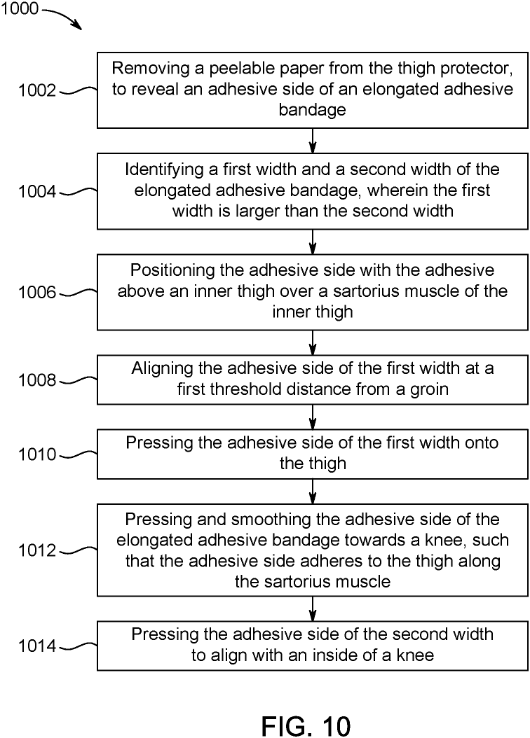

Referring now to FIG. 10, an exemplary flowchart of a method 1000 of using the thighs protector 100, 500, 700 is illustrated. The method 1000 is described with reference to the thighs protector 100 illustrated in FIGS. 1 through 4; the thighs protector 500 illustrated in FIGS. 5 and 6; and the thighs protector 700 illustrated in FIGS. 7 and 8.

At step 1002, the method 1000 includes removing a peelable paper from the thighs protector 100, 500, 700, to reveal an adhesive side (i.e., the first side 104 with the adhesive receiving surface 110) of the elongated adhesive skin dressing 102. At step 1004, the method 1000 includes identifying the first width ‘W₁’ and the second width ‘W₂’ of the elongated adhesive skin dressing 102, wherein the first width ‘W₁’ is larger than the second width ‘W₂’ (as best depicted in FIG. 3). At step 1006, the method 1000 includes positioning the adhesive side 104 with the adhesive (such as, the first adhesive 114) above the inner thigh 10 over the sartorius muscle 16 of the inner thigh 10 (as depicted in FIGS. 4, 6, 8). At step 1008, the method 1000 includes aligning the adhesive side 104 of the first width ‘W₁’ at the first threshold distance ‘D₁’ from the groin 18. At step 1010, the method 1000 includes pressing the adhesive side 104 of the first width ‘W₁’ onto the thigh (specifically, the inner thigh 10). At step 1012, the method 1000 includes pressing and smoothing the adhesive side 104 of the elongated adhesive skin dressing 102 towards the knee 12, such that the adhesive side 104 adheres to the thigh 10 along the sartorius muscle 16 (as depicted in FIGS. 4, 6, 8). At step 1014, the method 1000 includes pressing the adhesive side 104 of the second width ‘W₂’ to align with an inside of the knee 12 (as best depicted in FIG. 3).

With the present configuration, the thighs protector 100, 500, 700 acts as a sticker (and thus also referred to as thighs protector sticker, or thighs protector sticker as it protects both thighs, without any limitations) which may be easily applied to the inner thigh 10 by a human for preventing chafing due to friction, as caused by rubbing of inner thighs when performing any physical activity like walking or moving in a crowded environment. Because of the adhesive configuration, the thighs protector 100, 500, 700 is held against the inner thigh 10 once applied thereto. The thighs protector 100, 500, 700 further allows for its expansion and contraction to allow for expansion and contraction of the sartorius muscle 16 due to the physical activity, and thus is held in place while not discomforting the user. The thighs protector 100, 500, 700 is made of skin safe materials, is breathable, sweat-proof and the like.

The first embodiment of the present disclosure is illustrated with respect to FIG. 1 through FIG. 4. The first embodiment describes the thighs protector 100. The thighs protector 100 includes the elongated adhesive skin dressing 102 having the first side 104 including the adhesive pattern and the second side 106 including hydrophobic, friction free material, wherein the elongated adhesive skin dressing 102 is configured to cover at least a section of the inner thigh 10 of a human, wherein the elongated adhesive skin dressing 102 has the outer edge 108 which follows the outer extent 14 of the sartorius muscle 16 of the inner thigh 10 and tapers from the first width ‘W₁’ near a top of the inner thigh 10 to the second width ‘W₂’ at the inside of the knee 12, wherein the first width ‘W₁’ is larger than the second width ‘W₂’.

The present disclosure further describes the method 1000 of using the thighs protector 100, 500, 700, comprising removing a peelable paper from the thighs protector 100, 500, 700, to reveal the adhesive side 104 of the elongated adhesive skin dressing 102; identifying the first width ‘W₁’ and the second width ‘W₂’ of the elongated adhesive skin dressing 102, wherein the first width ‘W₁’ is larger than the second width ‘W₂’; positioning the adhesive side 104 with the adhesive above the inner thigh 10 over the sartorius muscle 16 of the inner thigh 10; aligning the adhesive side 104 of the first width ‘W₁’ at the first threshold distance ‘D₁’ from the groin 18; pressing the adhesive side 104 of the first width ‘W₁’ onto the thigh; pressing and smoothing the adhesive side 104 of the elongated adhesive skin dressing 102 towards the knee 12, such that the adhesive side 104 adheres to the thigh 10 along the sartorius muscle 16; pressing the adhesive side 104 of the second width ‘W₂’ to align with an inside of the knee 12.

Obviously, numerous modifications and variations of the present disclosure are possible in light of the above teachings. It is therefore to be understood that within the scope of the appended claims, the invention may be practiced otherwise than as specifically described herein.

1. A thighs protector, comprising:
an elongated adhesive skin dressing having a first side including an adhesive pattern and a second side including hydrophobic, friction free material,
wherein the elongated adhesive skin dressing is configured to cover at least a section of an inner thigh of a human,
wherein the elongated adhesive skin dressing has an outer edge which follows an outer extent of a sartorius muscle of the inner thigh and tapers from a first width near a top of the inner thigh to a second width at an inside of a knee, and
wherein the first width is larger than the second width.

2. The thighs protector of claim 1, further comprising:
a first adhesive on the outer edge of the first side of the elongated adhesive skin dressing;
a second adhesive on an inner extent of the first side, wherein the second adhesive has a lower tack strength than the first adhesive, wherein the first adhesive and the second adhesive are covered with a peelable paper.

3. The thighs protector of claim 1, further comprising:
at least one expansion band on the first side of the elongated adhesive skin dressing, wherein each expansion band includes at least one of:
a pleated material;
an adhesive pattern with a low tack adhesive region and a region without adhesive;
a pre-stressed material;
a folded material;
adhesive tracks; and
a tucked material.

4. The thighs protector of claim 1, further comprising:
a bumper extending to either side of a midline of the elongated adhesive skin dressing, the bumper including at least one of:
a thicker material;
layers of material; and
a filled channel.

5. The thighs protector of claim 4, wherein the filled channel includes one of hydroxyethyl cellulose, sodium polyacrylate, vinyl-coated silica gel and silicone.

6. The thighs protector of claim 1, further comprising:
an adhesive receiving surface on the first side of the elongated adhesive skin dressing; and
a hydrophobic, friction free material on the second side of the elongated adhesive skin dressing.

7. The thighs protector of claim 6, wherein the first adhesive is selected from the group comprising spirit gum, liquid latex, silicon rubber based adhesive, and medical dermal glue and the second adhesive is selected from the group comprising platinum silicone material, polyisoprene and polyisobutylene.

8. The thighs protector of claim 6, wherein the hydrophobic, friction free material is selected from the group comprising silk-like material, polyester material, and polypropylene (PP) fabric material.

9. The thighs protector of claim 1, further comprising:
wherein the elongated adhesive skin dressing tapers from the first width within a first threshold distance from a groin at a top of the inner thigh to the second width at a second threshold distance above a knee, where the first width is four times as large as the second width.

10. The thighs protector of claim 9, wherein the first threshold distance is selected from a range of 6 cm to 30 cm.

11. The thighs protector of claim 9, wherein the second threshold distance is selected from a range of 4 cm to 20 cm.

12. The thighs protector of claim 9, wherein the elongated adhesive skin dressing has a straight shape at the top of the inner thigh.

13. The thighs protector of claim 9, wherein the elongated adhesive skin dressing has a rounded shape at the second threshold distance above a knee.

14. A thighs protector, comprising:
an elongated adhesive skin dressing configured to cover at least a section of an inner thigh of a human,
wherein the elongated adhesive skin dressing has an outer edge which follows an outer extent of a sartorius muscle of the inner thigh and tapers from a straight first width within a first threshold distance from a groin at a top of the inner thigh to a rounded second width at an inside of the thigh at a second threshold distance above a knee, and
wherein the first width is larger as the second width.

15. The thighs protector of claim 14, wherein the first threshold distance is selected from a range of 0 cm to 8 cm.

16. The thighs protector of claim 14, wherein the second threshold distance is selected from a range of 2 cm to 8 cm.

17. The thighs protector of claim 14, further comprising:
an adhesive receiving surface on the first side of the elongated adhesive skin dressing; and
a hydrophobic, friction free material on a second side of the elongated adhesive skin dressing.

18. The thighs protector of claim 14, further comprising:
at least one expansion band on the first side of the elongated adhesive skin dressing, wherein each expansion band includes at least one of:
a pleated material;
an adhesive pattern with a low tack adhesive region and a region without adhesive;
a pre-stressed material;
a folded material;
an elastic material; and
a tucked material, wherein the pleated material, folded material, elastic material and tucked material are each configured to expand when the sartorius muscles stretches and retract when the sartorius muscle relaxes.

19. The thighs protector of claim 18, further comprising:
wherein the low tack adhesive region is configured to release when the sartorius muscles stretches and reattach when the sartorius muscle relaxes.

20. A method of using a thighs protector, comprising:
removing a peelable paper from the thighs protector to reveal an adhesive side of an elongated adhesive skin dressing;
identifying a first width and a second width of the elongated adhesive skin dressing, wherein the first width is larger than the second width;
positioning the adhesive side with the adhesive above an inner thigh over a sartorius muscle of the inner thigh;
aligning the adhesive side of the first width at a first threshold distance from a groin;
pressing the adhesive side of the first width onto the thigh;
pressing and smoothing the adhesive side of the elongated adhesive skin dressing towards a knee, such that the adhesive side adheres to the thigh along the sartorius muscle; and
pressing the adhesive side of the second width to align with an inside of a knee.
